(19) United States
(12) Patent Application Publication
(10) Pub. No.: US 2024/0279598 A1
(43) Pub. Date: Aug. 22, 2024

(54) BIOREACTOR SYSTEM WITH ENHANCED CELL HARVESTING CAPABILITIES AND RELATED METHODS

(71) Applicant: UNIVERCELLS TECHNOLOGIES SA, Nivelles (BE)

(72) Inventors: Jean-Christophe Drugmand, Wavre (BE); Bastien Mairesse, Uccle (BE); José Castillo, Brussels (BE); Christophe Dumont, Haine-Saint-Pierre (BE); Sebastien Rodriguez, Ecaussines-Lalaing (BE); Antoine Hubert, Nivelles (BE); Tania Pereira Chilima, Nivelles (BE)

(21) Appl. No.: 18/566,901

(22) PCT Filed: Jun. 3, 2022
(86) PCT No.: PCT/EP2022/065264

§ 371 Date: Dec. 4, 2023

Related U.S. Application Data
(60) Provisional application No. 63/330,981, filed on Apr. 14, 2022.
(60) Provisional application No. 63/323,308, filed on Mar. 24, 2022.
(60) Provisional application No. 63/310,753, filed on Feb. 16, 2022.
(60) Provisional application No. 63/295,673, filed on Dec. 31, 2021.
(60) Provisional application No. 63/196,337, filed on Jun. 3, 2021.

(51) Int. Cl. C12N 5/00; C12M 1/00; C12M 1/12; C12M 1/26; C12M 1/34

(52) U.S. Cl. CPC C12N 5/0068; C12M 25/18; C12M 33/08; C12M 41/44; C12M 47/02; C12N 2509/00

(57) ABSTRACT

Systems and methods for harvesting cells include a bioreactor with a structure for cell entrapment/adherence and growth, such as a fixed bed structure. A cell harvest mechanism is adapted to agitate the bioreactor, such as by vibrating or shaking the fixed bed, and to move a liquid level relative to the structure, such as by repeatedly flushing and filling the bioreactor. A cell detaching solution is provided to the bioreactor, such as an enzymatic cocktail for the detachment of cells without producing clumps or aggregates. The bioreactor may comprise a preculture vessel upstream of another bioreactor.

### TECHNICAL FIELD

This document relates generally to a bioreactor system with enhanced cell harvesting capabilities and related methods.

### BACKGROUND

Certain high cell density bioreactors used for biomanufacturing include a structure, such as a fixed (whether structured or packed) bed, for promoting cell entrapment/adhesion and growth. The arrangement of the material of the fixed bed affects local fluid, heat, and mass transport. In many cases, it is very dense to maximize cell cultivation in a given space.

For certain biomanufacturing applications, the cells that grow in the fixed bed are themselves harvested from the bioreactor after the growth stage. This can be the case when the cell harvest is to be used as a seed train for expansion of cells for the inoculation of another (e.g., production) bioreactor, or when he cells themselves are the product of interest (e.g., to produce a cell bank or for cell therapy applications). In order to harvest the viable cells from the fixed bed, chemical agents, such as the trypsin enzyme, may be used. However, this alone often results in a limited amount of cell detachment, often due to the densely packed nature of the fixed bed material in a typical bioreactor, which makes it more difficult to circulate the chemical agent throughout the bed and increase the yield of cells harvested.

Cells also attach to the fixed bed through small cable-like proteins called integrins. Moreover, cells produce other proteins, like collagen and glycosaminoglycans, which form an extracellular matrix, similar to a net. Even when a detachment solution such as trypsin is used to achieve detachment of cells from the structure, these proteins can cause the harvested cells to clump together. This may be undesirable for purposes of recovery and may lead to lower-than-desired cell yields.

Thus, a need is identified for a manner of improving the yield of cells harvested from a bioreactor.

### SUMMARY

According to one aspect of the disclosure, a method of harvesting cells is provided. The method includes providing a bioreactor including a fixed bed structure capable of cell entrapment or adherence and cell growth, adding cells to the bioreactor via media, and allowing the cells to become entrapped and/or adhere to the fixed bed structure and grow within the bioreactor. The method further includes agitating the bioreactor and moving a liquid level relative to the fixed bed structure and introducing a cell detaching solution comprising an enzymatic cocktail into the bioreactor, wherein a portion of the cells are detached from the fixed bed structure without clumps or aggregates in the portion of the cells.

In one embodiment, the agitating and moving steps are done simultaneously. The moving step may comprise at least partially draining the bioreactor of liquid, such as by moving the liquid level from adjacent a top of the fixed bed structure to adjacent a bottom of the fixed bed. The moving step may comprise adding liquid to the bioreactor, such as for example by adding additional cell detaching solution to the bioreactor. The moving step may comprise moving the structure for cell entrapment/adherence and growth relative to the bioreactor to move a location of the liquid level.

The liquid level may be located above the fixed bed structure prior to the moving step, which may involve raising and lowering the liquid level (such as from the top of the fixed bed structure to the bottom of the fixed bed structure, for example), a plurality of times (but it could also be only one time). The agitating step may comprise vibrating the bioreactor. The step of introducing may comprise enzymes that cleave integrins and different enzymes that cleave an extracellular matrix as the enzymatic cocktail.

According to a further aspect of the disclosure, a system for harvesting cells is provided. The system comprises a bioreactor including a structure for cell entrapment/adherence and growth, a cell harvest mechanism adapted to agitate the bioreactor and to move a liquid level relative to the structure, and a vessel including a cell detaching solution in fluid communication with the bioreactor. The cell detaching solution comprises an enzymatic cocktail for the detachment of a portion of the cells from the structure for cell entrapment/adherence and growth without producing clumps or aggregates in the portions of the cells once detached.

In one embodiment, the structure for cell entrapment/adherence and growth comprises a fixed bed, such as a 3-D printed fixed bed. The structure for cell entrapment/adherence and growth comprises a fixed bed having a plurality of cell immobilization layers, such as arranged in a stack or a spiral configuration, and either in direct contact or with a spacing between adjacent layers. The cell harvest mechanism comprises a device for vibrating or shaking the bioreactor (in particular to the structure the structure for cell entrapment/adherence and growth) and/or a pump. The cell harvest mechanism may form part of a docking station for the bioreactor, which may comprise a harvest vessel for harvesting cells for introduction to another bioreactor.

In these or other embodiments, the bioreactor may be tilted relative to a horizontal plane to facilitate draining of liquid from the structure for cell entrapment/adherence and growth. A compactor may be provided for compacting the structure for cell entrapment/adherence and growth, either internal or external thereto. The enzymatic cocktail includes enzymes that cleave integrins and different enzymes that cleave an extracellular matrix.

The cell harvest device may comprise an actuator for moving the structure for cell entrapment/adherence and growth relative to the bioreactor to move a location of the liquid level. A controller may be provided for controlling the cell harvest mechanism to agitate the bioreactor and move a liquid level relative to the structure for cell entrapment/adherence and growth. The controller may be adapted for controlling delivery of the enzymatic cocktail to the bioreactor.

A further aspect of the disclosure pertains to a system for harvesting cells including a bioreactor including a structure for cell entrapment/adherence and growth, an agitator adapted to agitate the bioreactor, an actuator for moving a liquid level relative to the structure for cell entrapment/adherence and growth, and a vessel including a cell detaching solution in fluid communication with the bioreactor. The cell detaching solution comprises an enzymatic cocktail for the detachment of cells from the structure for cell entrapment/adherence and growth without producing clumps or aggregates.

In one embodiment, the agitator comprises a vibrator. The actuator may comprise a linear actuator and/or a pump. A controller may also be provided for controlling the actuator and/or the agitator.

Yet a further aspect of the disclosure pertains to a system for harvesting cells. The system includes a bioreactor including a structure for cell entrapment/adherence and growth. A cell harvest mechanism is adapted to adapted to agitate the bioreactor while filling and flushing the bioreactor with liquid. A vessel includes a cell detaching solution in fluid communication with the bioreactor.

In one embodiment, the cell detaching solution comprises an enzymatic cocktail for the detachment of cells without producing clumps or aggregates. The cell harvest mechanism may comprise a device for applying vibratory energy to the bioreactor and, in particular, to the structure for cell entrapment/adherence and growth, such as by vibrating or shaking the bioreactor and/or the structure, and/or a device to partially or completely fill, empty and flush the bioreactor. The fill, empty and flush may device comprise one or more pumps. The cell harvest mechanism may form part of a docking station for the bioreactor.

Still another aspect of the disclosure relates to a method of detaching cells from a fixed bed bioreactor, comprising: adding to the fixed bed bioreactor an enzymatic cocktail for the detachment of cells without producing clumps or aggregates; and adjusting a position of a liquid level in the fixed bed bioreactor while vibrating the bioreactor. The adjusting step may comprise filling and flushing the bioreactor with liquid, including by repeatedly filling and flushing the bioreactor with liquid. The method may further include the step of delivering detached cells from the bioreactor to another bioreactor. Still further, the method may comprise tilting the bioreactor and/or compacting a fixed bed in the bioreactor. The adjusting step may comprise moving the fixed bed relative to the bioreactor.

Another disclosed aspect is a method of detaching cells in a bioreactor. The method includes vibrating the bioreactor and tilting and draining the bioreactor. The vibrating, tilting, and draining steps may be performed concurrently.

Furthermore, this disclosure relates to a system for harvesting cells. The system comprises a bioreactor including a fixed bed for adherent cell growth, and a compactor for compacting the fixed bed to facilitate the removal of cells. A vibrator may be provided for vibrating the bioreactor or the fixed bed. The compactor may be located within the fixed bed or external to the fixed bed.

Still further, this disclosure relates to a system for harvesting cells. The system comprises a preculture vessel including a structure for adherent cell growth and a vibrator adapted to vibrate the bioreactor in order to detach cells from the structure. A bioreactor downstream of the preculture vessel is for receiving the detached cells. A pump may also be provided for pumping liquid to or from the preculture vessel so to move a liquid level relative to the structure, and a controller may be provided for controlling the pump, as well as possibly the vibrator.

### BRIEF DESCRIPTION OF THE DRAWING FIGURES

FIG. 1 schematically illustrates a bioreactor system according to one aspect of the disclosure.

FIG. 2 shows more detailed example of a bioreactor system according to another aspect of the disclosure.

FIG. 2A illustrates an example of a connector for connecting the bioreactor to an agitator, such as a vibrating table.

FIG. 2B is a flow diagram illustrating an exemplary use of the bioreactor system according to the disclosure.

FIG. 3 depicts a bioreactor and shows the manner in which liquid is moved, or pulsed, within an associated bed during the application of vibration to improve the cell harvest.

FIGS. 3A, 3B, 3C, 3D, 3E, and 3F illustrate various other forms of bioreactor arrangements that may benefit from the various inventive aspects disclosed herein.

FIG. 4 schematically illustrates a further embodiment of a bioreactor system according to another aspect of the disclosure.

FIGS. 4A, 4B, and 4C illustrate a bioreactor integrated into a docking station, which includes a vibration table.

FIG. 5 illustrates different manners of applying mechanical energy to drain a fixed bed.

FIGS. 6 and 7 illustrate tilting of a bioreactor during draining to aid in fluid recovery.

FIGS. 8, 9, and 10 illustrate various embodiments of compactors for compacting a fixed bed.

FIGS. 11-16 illustrate a bioreactor including a dynamic fixed bed.

FIGS. 17-19 illustrate examples of various aspects of control for a bioprocessing operation.

FIGS. 20-24 illustrate details of exemplary experiments conducted according to various aspects of this disclosure.

### DETAILED DESCRIPTION

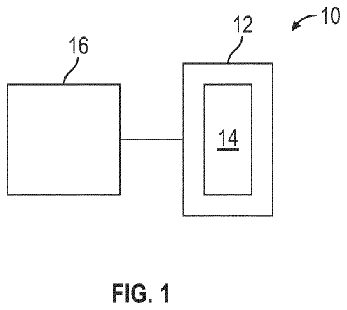

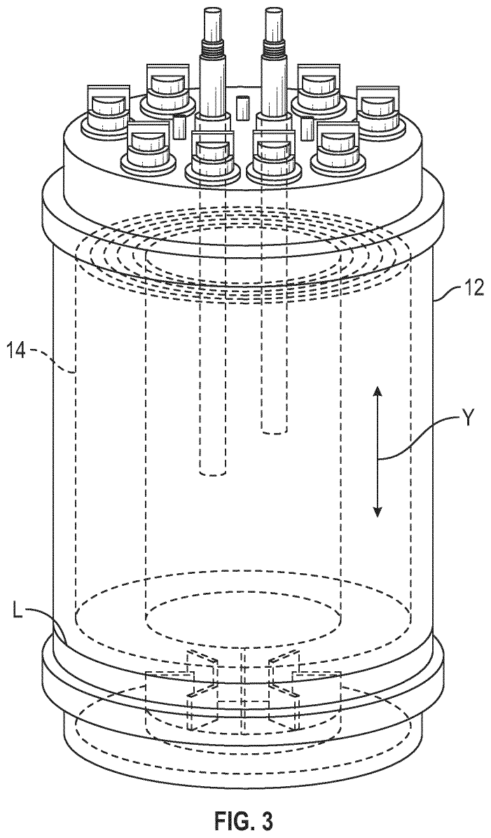

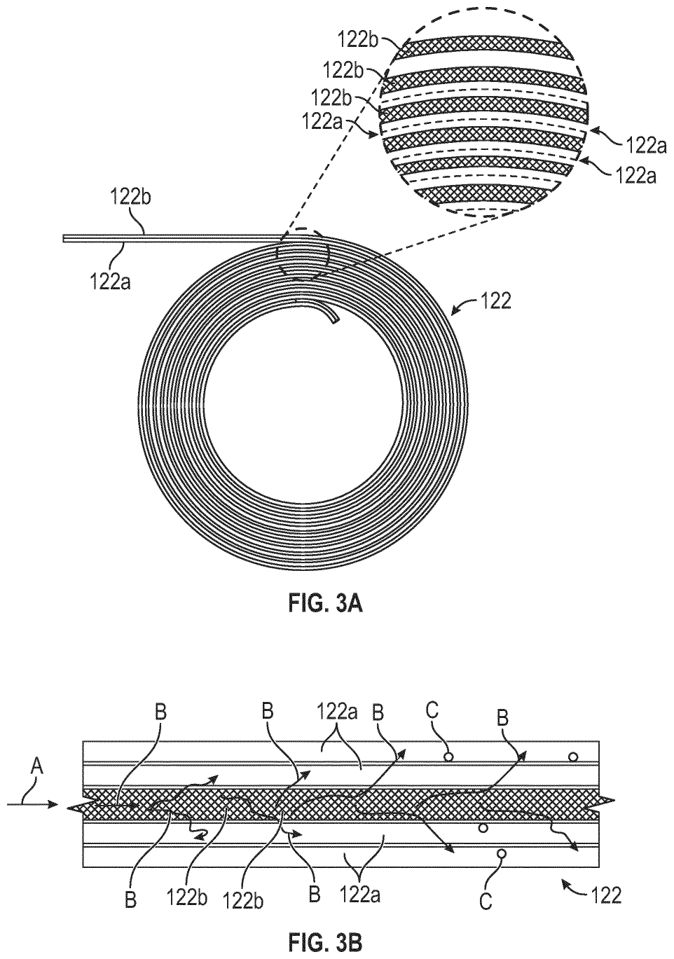

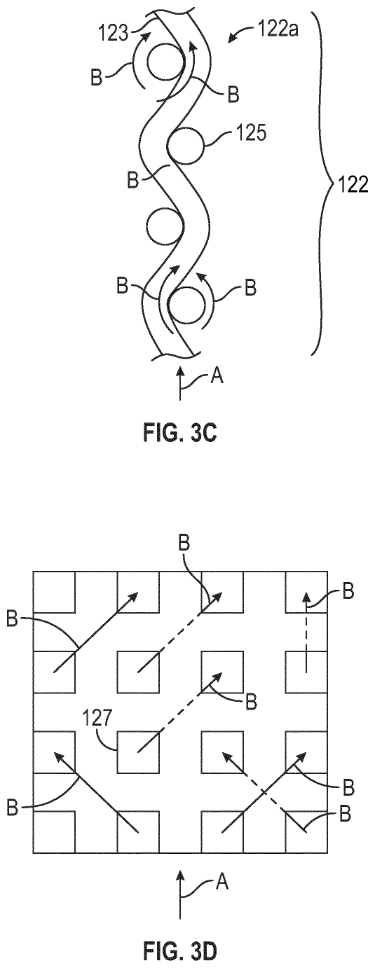

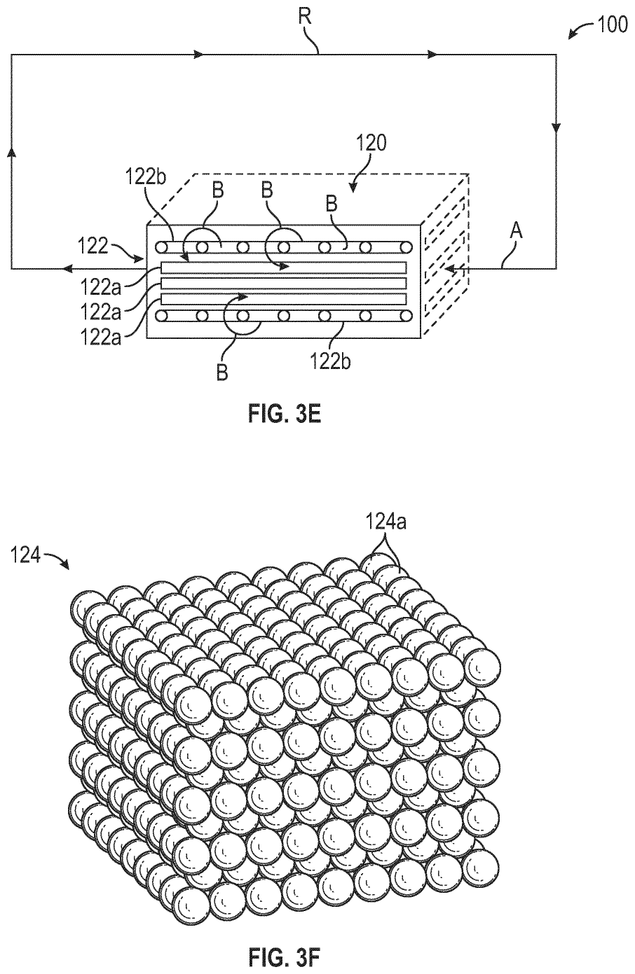

In one aspect, this disclosure pertains to a bioreactor with enhanced cell harvesting capabilities. With reference to FIG. 1, this may be achieved by way of a system 10 including a bioreactor 12, such as one including a structure for adherent or suspension cell growth, such as a fixed bed 14. The fixed bed 14 may comprise, for example, a structured fixed bed, a 3-D printed matrix, a bed comprised of a woven or non-woven material(s), such as for example one or more sheets of such a material in direct contact or with interposed spacers, beads, hollow fibers, or any other suitable cell culture structure for promoting adherent cell growth or cell growth via entrapment (see, e.g., the description of FIGS. 3A-3F below). The bed 14 may be designed in any desired shape, orientation, or form, including for example 3D porous monoliths, stacked layers (see, e.g., U.S. Pat. No. 11,111,470, the disclosure of which is incorporated herein by reference), parallel layers arranged vertically, layers arranged in a spiral or wound configuration, or packed beds (see, e.g., U.S. Pat. No. 8,137,959, the disclosure of which is incorporated herein by reference).

The system 10 further includes a cell harvest mechanism 16 for improving the yield of cells harvested from the bioreactor 12. The cell harvest mechanism 16 may be integrated with the bioreactor 12 and/or may be engaged or used only when cell harvesting is desired, such as by way of connection to or interaction with the bioreactor. As outlined further in the description that follows, the harvest mechanism 16 may also be built into a docking station for the bioreactor 12 or provided outside of an integrated system.

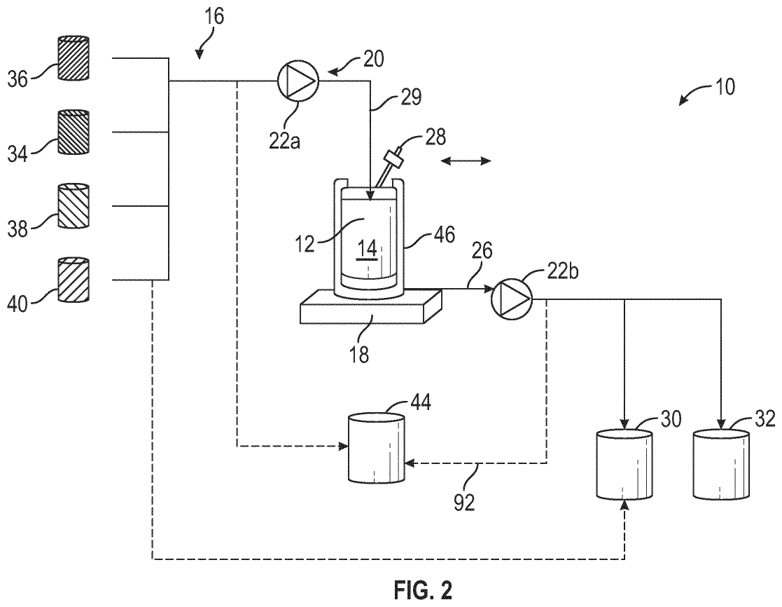

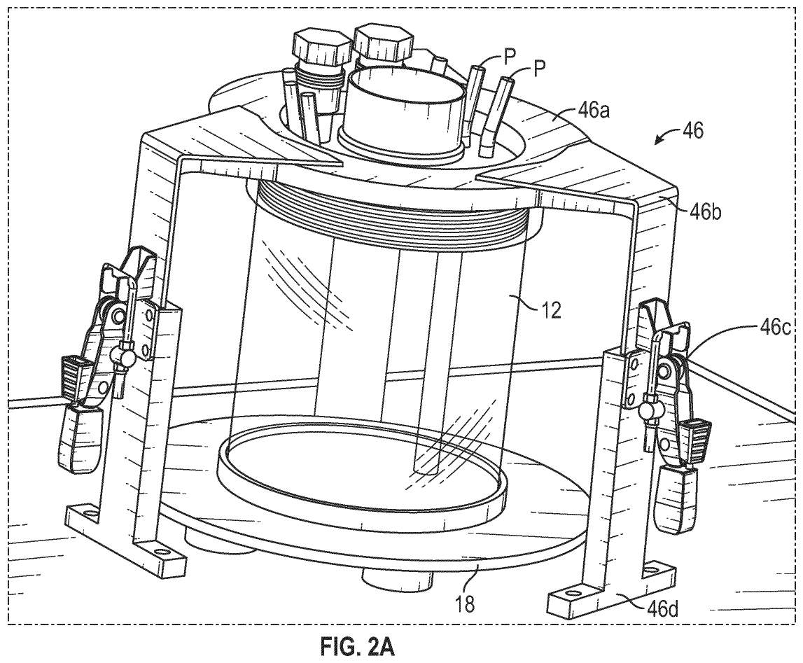

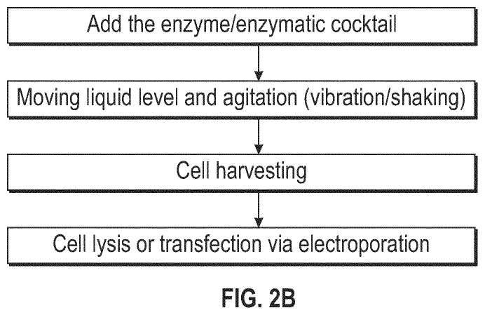

In one example, as shown in FIG. 2, the cell harvesting mechanism 16 may include a first device 18 for agitating the bioreactor 12 and a second device 20 for moving or changing a liquid level within the bioreactor 12, such as in one example by filling the bioreactor 12 with a liquid chemical cocktail (see below) so that the liquid level is at or above the top portion of the fixed bed and draining the bioreactor 12 so as to move the liquid level from the fill level to the bottom of or below a bottom of the fixed bed 14. Alternatively or additionally, the second device 20 may create a reciprocating, or “back and forth,” movement of a portion of the liquid between the inside and the outside of the fixed bed bioreactor 12. This back and forth movement of the liquid can be created by an actuator, such as one or more pumps, for creating a pulsing action where liquid is partially drained and then partially introduced into the bioreactor, or it can involve a complete draining and refilling of the bioreactor 12. For this purpose, the bioreactor 12 may be associated with an inlet 24, an outlet or drain 26, each of which may be associated with suitable pumps 22a, 22b, and a vent 28. The bioreactor 12 may comprise a rigid vessel, or may comprise a disposable or single use vessel or bag.

The first device 18 may be, for example, any means of applying agitating energy to the bioreactor 12 or to the fixed bed 14. The portion of the bioreactor 12 to which the energy is applied may include any part of it as long as it results in vibration of the fixed bed 14 in a manner sufficient to cause cell detachment. The device 18 may comprise, for example, an agitator in the form of a vibrational table, a vortex device, a shaker, or another device for applying mechanical energy to the bioreactor and/or the structure for adherent cell growth/entrapment, such as the fixed bed 14. The device 18 may be internal to or external to the bioreactor 12. The vibrating motion can be oscillating, reciprocating, or periodic, either harmonic or random. The frequency may be 20-100 Hertz or, more specifically, 50-80 Hertz. The amplitude may be low, such as 0.5-5.0 millimeters or, more specifically, 2-3 millimeters.

The second device 20 may comprise, for example, one or more liquid transfer devices, such as two-way or reversible pumps 22a, 22b or other means to transmitting fluid to and from the bioreactor 12. The second device 20 may cause the fluid drained during a drain mode to be recirculated into the bioreactor 12 during fill mode, or fresh fluid may be introduced into the bioreactor 12 during such fill mode. The second device 20 may also perform only a draining, emptying, or filling cycle, and may be done using only one such cycle (e.g., one draining or filling) or multiple cycles.

The second device 20 may be integrated with the first device 18 so that these devices work in tandem or in parallel. Alternatively, the devices 18, 20 may be portions of a single device. In either case, a controller (e.g., computer or processor) may be provided that manages an algorithm or process for combined agitation and liquid movement back and forth to and from the bioreactor 12 in an automated fashion or as a result of operator commands.

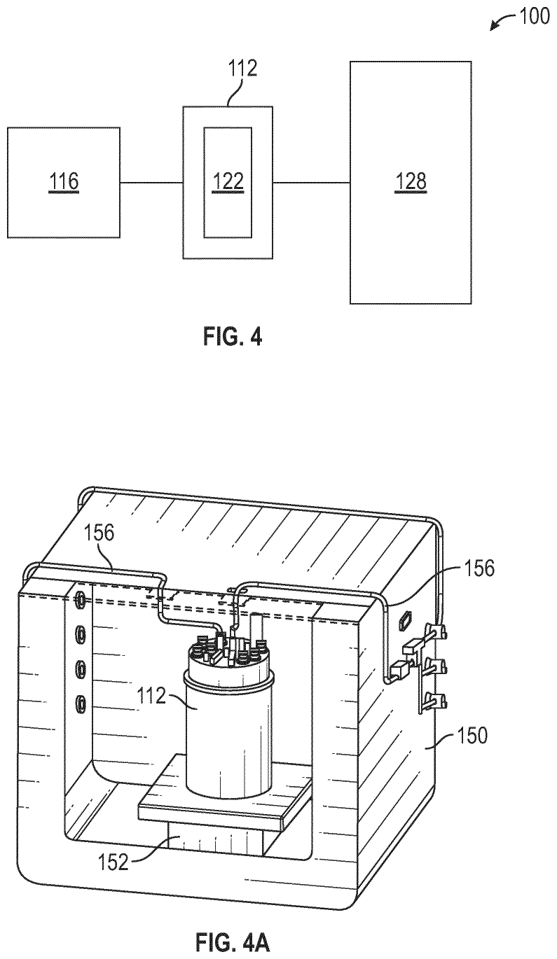

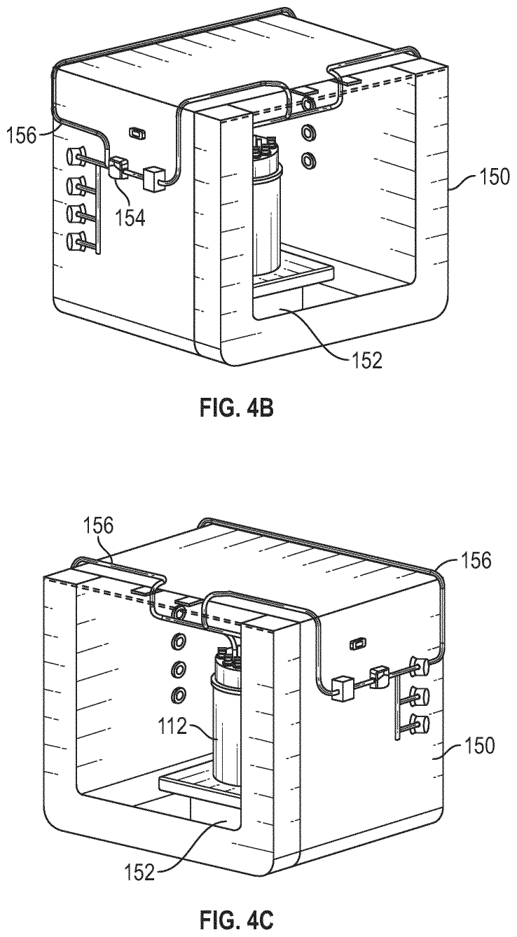

The system 10 may further include a harvest vessel 30, a waste vessel 32, and a supply vessel 34 containing a cell detaching solution, such as one comprising an enzyme (including possibly an enzymatic cocktail, as outlined further in the description that follows), each in fluid communication with the bioreactor 12. Optional vessels 36, 38, 40 for supplying rinsing and inactivation solutions may also be provided in fluid communication with the bioreactor 12. For example, vessel 34 may provide an enzymatic and/or chemical cell detaching solution (e.g., Trypsin/PBS/EDTA, which may be warmed to 37 degrees Celsius), vessel 36 may provide an optional rinsing solution (e.g., PBS/EDTA), vessel 38 may include another form of optional rinsing buffer solution (e.g., PBS), and vessel 40 may provide an optional inactivation solution (e.g., STI serum). Any or all of these vessels (and solutions therein) may optionally be agitated, and may be part of a recirculation loop 42 to allow a recirculation with the bioreactor 12 (because the amount of enzyme (e.g., trypsin) needed to detach the cells could higher than the nominal volume of the bioreactor), possibly with a reservoir 44. The harvest vessel 30 in particular may be agitated and optionally controlled in temperature to prevent the harvested cells from settling, such as before being used to inoculate another bioreactor, which may be located upstream (see, e.g., FIG. 4). The system 10 may also be adapted to preheat the detaching solution and/or to maintain the temperature of the cell detaching solution (usually at 37° C.).

Using the above system, the agitation applied to the bioreactor 12 combined with movement of the liquid inside the bioreactor 12 are used to enhance the cell harvest. For example, the system 10 could vibrate, pulse or shake the bioreactor vessel while circulating the detaching solution via external pumping with the pulsing, or back and forth, liquid movement. Alternatively, the detaching solution can be moved internally by using internal circulation within bioreactor (such as via an agitator), or by using external recirculation-circulation or perfusion). For example, the vibration may be at a selected frequency (e.g., 20-300 Hz, including for example 60-80 Hz) and the pulsing of the liquid applied for multiple cycles (e.g., between 1 and 10, and at a flow rate of between 0.1-5 L/min).

Such agitation results in a maximum energy transfer at the liquid level adjacent to the gas phase of the bioreactor 12. By dynamically adjusting (e.g., raising and/or lowering, note arrow Y in FIG. 3) the liquid level within the bioreactor 12 and along the fixed bed 14 during the vibrating/shaking/agitating action (note line L in FIG. 3 near the bottom portion of fixed bed 14 of bioreactor 12), such as by using the second device 20 (e.g., pump), the cells are more effectively detached from the material of the fixed bed. Consequently, the yield or harvest of cells from the bioreactor 12 is increased in an easy and relatively inexpensive manner, and without significant added cost or complexity.

In order to maintain the integrity of the bioreactor 12 during the agitation, the bioreactor 12 may be attached to the system 10 and, in particular, the first device 18, using a connector 46. The connector 46 may comprise a mechanical structure for coupling the device 18 to the bioreactor 12, and should be sufficiently rigid in order to transfer the mechanical energy to the bioreactor 12.

In order to protect against mechanical damage, the connector 46 should be properly fitted to the bioreactor 12, and should maintain and protect any fragile part of the bioreactor present (e.g., pH and DO probes P) to prevent damage.

In the illustrated example shown in FIG. 2A, the connector 46 includes an annular part 46a for engaging the lid or cover of the bioreactor 12 with depending portions 46b. These depending portions 46b are releasably connected (such as by clamps 46c) to supports 46d. The supports 46d are directly attached to the device 18 in a manner that allows for the transmission of mechanical energy while maintaining security for the bioreactor 12 during agitation.

As examples, the system 10 may operate as follows:
In batch mode with concentrated enzyme (such as trypsin) (amount of enzyme equivalent to the nominal volume of the bioreactor 12);
In perfusion mode (inlet and outlet) for the enzyme; or
In recirculation (such as using an external loop to circulate enzyme).

As a further aspect, and with reference to the flow diagram of FIG. 2B, one exemplary use of the system 10 shown in FIG. 2 may involve performing the following steps that follow the inoculation and cell growth phase:
1. After completing a growth phase of the bioreactor with cells entrapped inside the bed and grown, empty the bioreactor 12 using the drain (e.g., bottom line), such as to a waste vessel (e.g., vessel 32 in FIG. 2).
2. Optionally rinse the bioreactor 12 by adding a rinsing buffer, mixing it, and emptying the rinsing buffer (this step may be performed several times—e.g., between 1 to 5 times- and can be also done in perfusion by continuously filling and emptying the bioreactor).
3. Add the detaching solution to the bioreactor so that the liquid level thereof reaches or exceeds the height (or length) of the fixed bed, such as one including the trypsin enzyme, potentially preheated and diluted in an appropriate buffer (e.g., potentially containing chelators).
4. Optionally wait a period time (e.g., 1-60 min), potentially maintaining the temperature of liquid in a recirculation loop from 8° C. to 37° C.
5. Moving the liquid level, such as for example by draining or otherwise pumping the solution out of the bioreactor (optionally performing back and forth circulation of the detachment solution to the bioreactor 12 to fill and empty the fixed bed 14 (e.g., a few cycles—from 1 to 10 times, at a flow rate between 0.1 and 5 L/min) or optionally circulating the detachment solution (such as via recirculation loop)). During this step, mechanical energy (such as from vibration shaking or other agitation) is be applied to the bioreactor 12, combined with the movement of the liquid level relative to the bed 14. The draining/emptying of the solution may occur once or, or the filling/emptying can be implemented more times to more effectively detach the cells.
6. Harvest cells from bioreactor 12 (e.g., empty using the drain line).
7. Optionally rinse the bioreactor 12 and combine the rinse with the harvest.
8. Optionally add enzymatic inhibitor to the harvest (using serum, soy trypsin inhibitor, etc.). As noted above, the volume of the detaching enzyme may be higher than the nominal volume of the bioreactor 12. For example, in the case of a bioreactor 12 having a 30 m² bed and a classical trypsin concentration of 0.023 ml/cm² (150 ml/6600 cm², recommendation for CS/CF10), the amount of trypsin to be added into the bioreactor should be about 7 L. Hence, the trypsinization should be done in recirculation, in perfusion or in several steps (steps 3 to 6—see above). For a nominal volume of around 3 L, the trypsinization could be performed in two steps with the benefit of a rinsing step.

Still a further aspect of the disclosure pertains to the concept of using a cell detachment solution in the form of an enzymatic cocktail in connection with cell harvesting from a bed bioreactor, per any aspect of this disclosure or otherwise. The proposal is to introduce to the fixed bed during cell harvesting an enzymatic cocktail comprising a mix of different enzymes that cleave integrins and the extracellular matrix allow for the detachment of cells in high yields without the aggregation issue faced with using a single enzyme, such as trypsin. As an example, the enzymatic cocktail may comprise: (1) a serine protease, such as trypsin (preferential cleavage: Arg-|-Xaa, Lys-|-Xaa); and/or (2) one or more of the following: (a) Chymotrypsin (preferential cleavage: Leu-|-Xaa, Tyr-|-Xaa, Phe-|-Xaa, Met-|-Xaa, Trp-|-Xaa, Gln-|-Xaa, Asn-|-Xaa; (b) elastase (preferential cleavage: Hydrolysis of proteins including elastin, collagen types III and IV, fibronectin and immunoglobulin A, generally with bulky hydrophobic group at P1); (c) collagenase I (preferential cleavage: Cleavage of the triple helix of collagen at about three-quarters of the length of the molecule from the N-terminus, at 775-Gly-|-Ile-776 in the alpha-1(I) chain); and/or (d) cysteine protease (e.g. papaine, etc.). Commercially available examples of enzymatic cocktails (containing different enzymatic cocktails with the following activities: Trypsin, Chymotrypsin, Elastase, Collagenase type I) are Accutase and Accumax (distributed by Innovative Cell Technologies). DNases could also be used as a cleaving/anti-aggregating agent.

The use of such enzymatic cocktails prevents forming of clumps, aggregates, or having a single enzyme solution that may be too aggressive for the cells (e.g., one that decreases cell viability, etc.). The enzyme cocktail can be used in a single solution or added sequentially to the bioreactor, including in batch mode with mix of enzymes (amount of enzyme equivalent to the nominal volume of the bioreactor), perfusion (inlet and outlet) mode, or recirculation mode.

As an example of a protocol that may be used during cell harvesting involves the following steps that follow the inoculation and cell growth phase:
1. Empty the bioreactor 12 using the drain (e.g., bottom line) to the waste vessel.
2. Optionally rinse by addition the rinsing buffer, apply mixing, and empty the rinsing buffer (this step could be done multiple times, and could be also done in perfusion mode).
3. Add the detachment solution comprising an enzyme cocktail (mix of enzymes) to the bioreactor so that its liquid level is at or above the height (or length) of the fixed bed, which may be preheated.
4. Optionally wait a period of time (e.g., 2-30 min) while maintaining the temperature at from 22° C. to 37° C. (this step can be done both in batch and recirculation modes).
5. Move the liquid level of the solution so that it travels through the fixed bed and until it is below or beyond the other end of the fixed bed, or move the liquid level such as by complete recirculation to fill and empty the bed (from 1 to 10 cycles), optionally circulate the enzyme cocktail (such as via recirculation loop) and apply vibration at 10-200 Hz (shaking/agitation) at low amplitude on the bioreactor 12 to detach the cells combined with back and forth pulsing movement inside the fixed bed 14.
6. Harvest cells from the bioreactor (e.g., using the drain line).
7. Rinse the bioreactor 12 and pool the rinse product together with the harvest.
8. Optionally, add enzymatic inhibitor to the harvest (using serum, soy trypsin inhibitor, chelating agents, dilution, etc. . . . ).

A further aspect of this disclosure also includes the recovery of cells from the fixed-bed bioreactor using enzymes or a cocktail of enzymes, combined with a mechanical energy applying (e.g., vibrating) device, preferably before and during the liquid level moving step above. After recovery or harvest, the cells such may be lysed outside of the bioreactor using reagents or mechanical action (e.g. microfluidizer, homogenizer) in order to release intracellular or cell-associated viruses. This also includes the recovery of the cells using enzymes or a cocktail of enzymes combined with a vibrating device from the fixed-bed bioreactor for transfection via electroporation in a second reactor.

Any or all of the aspects of this disclosure may be applied alone or in combination to other forms of fixed beds. For example, with reference to FIGS. 3A-3B, the fixed bed 14 may comprise a structured fixed bed 122 for cells (adherent or otherwise) including one or more cell immobilization layers 122a, which may be wound into a spiral form as shown. The one or more layers 122a provide a tortuous channel of flow (arrow B) from a linear or regular inflow (arrow A) without using additional spacer layers (but such may be used, if desired). This may be achieved, for example, as shown in FIG. 3C by providing a layer of woven fibers or filaments 123, 125 that disrupt the flow.

FIG. 3D shows that such a result may be achieved using a non-woven material as the cell immobilization layer 122a. This may be achieved by forming the layer 122a as a reticulated arrangement (such as by 3-D printing) with openings 127 through which liquid may pass and return again, thus forming the tortuous channels that again promote homogeneity and also serve to further shear or divide any bubbles present in the liquid. This function may again be achieved with or without added spacer layers being present.

The orientation of the structured fixed bed 122 may be other than as shown in a bioreactor 12 where the flow is arranged vertically (bottom to top, in the example provided by FIG. 3). For example, as shown in FIG. 3E, a horizontally arranged bioreactor 100 may include a first chamber 120 that includes a structured fixed bed 122 comprised of one or more horizontally arranged material layers. These one or more layers may comprise a woven or reticulated material, as per FIGS. 3C and 3D, but as illustrated in FIG. 3E, may comprise one or more cell immobilization layers 122a (three shown, but any number may be present) sandwiched by adjacent spacer layers 122b (vertical spacing exaggerated for purposes of illustration). The flow is thus arranged from side-to-side (left to right or right to left), with the material layer(s) (spacer or otherwise) providing for the channels for creating the tortuous flow (arrows B) from a linear or regular inflow (arrow A) and thus serving to further divide any bubbles present in the liquid. The pumping action may be provided by an agitator or other pump at the entrance end of the chamber 120, and a return path provided at the exit end, as schematically illustrated by path R. Additional spacer layers may also be provided between the cell immobilization layers 122a, if desired.

In another possible embodiment, and with reference to FIG. 3F, the structured fixed bed 122 comprises a three-dimensional (3D) monolith matrix 124 in the form of a scaffold or lattice formed of multiple interconnected units or objects 124a, which have surfaces for cell adhesion. The matrix 124 may include a tortuous path for fluid and cells to flow therethrough when in use. In some embodiments, the matrix may be in the form of a 3D array, lattice, scaffolding, or sponge. The matrix 124 is preferably single use in nature to avoid the cost and complexities involved in cleaning according to bioprocessing standards.

According to a further aspect of the disclosure, and with reference to FIG. 4, a system 100 includes a bioreactor for serving as a preculture vessel 112 (e.g., a seed train) for use in producing cells for use in seeding another vessel, such as a production bioreactor 128. The preculture vessel 112 may comprise a structured fixed bed 122, such as one that comprises one or more spirally wound layers, as shown in FIG. 3A. Alternatively, the fixed bed 122 could be designed in a horizontally stacked form, as shown in FIG. 3E (with flow in the horizontal, rather than vertical direction), or any other known form including the other forms disclosed herein.

As indicated in FIGS. 4A, 4B, and 4C, the bioreactor for serving as a preculture vessel 112 (or any other bioreactor described herein) may be associated with a docking station 150. This station 150 may include an integrated vibration table 152 upon which the vessel 112 may rest. An integrated pump 154 and transfer lines 156 for liquid delivery as part of a cell culturing system, including with upstream and downstream processing using additional bioreactor(s), as desired, or possibly for delivering a cell detachment solution to the preculture vessel.

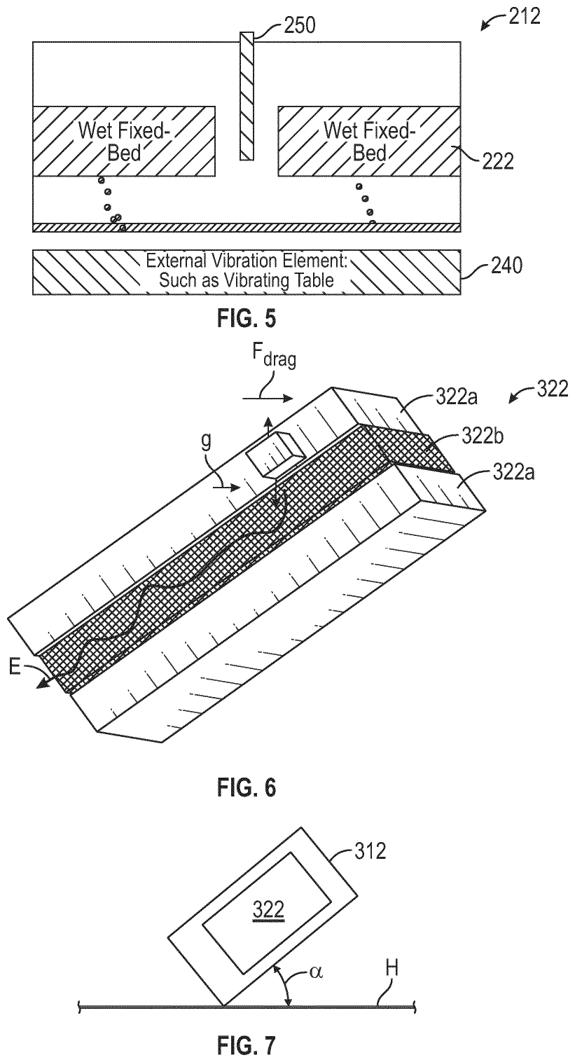

According to still a further aspect of the disclosure, and with reference to FIG. 5, a system 200 may be provided including a bioreactor 212 including a fixed bed 222 and an agitator. The agitator may comprise either or both of an external vibrator, such as a table 240 on which the bioreactor 212 is placed, or instead an internal vibrator 250 placed within the bioreactor 212 to transmit vibrations to the fixed bed 222. Because the fixed bed 222 is typically formed of a hydrophilic material, it tends to retain liquid during harvesting of the cells. Applying vibrations before, during or after draining of the bioreactor 212 during cell harvesting may cause any liquid within the fixed bed 222 to be released, thereby potentially further improving the recovery of any cells remaining in the trapped liquid.

With reference to FIGS. 6 and 7, yet a further aspect of the disclosure pertains to the concept of tilting or slanting a bioreactor 310 and, in particular, a fixed bed 322 of the bioreactor, in order to improve the liquid recovery during harvesting. By tilting or slanting the fixed bed 322, gravity is following the vertical direction, the drag force (F_Drag) is decreased in magnitude. As the magnitude of gravity applied on the volume of liquid is the same, but the residual F_Drag is decreased, the gravity magnitude is higher than the drag force, F_Drag. Then, the volume of liquid can escape from the cell immobilization layer 322a (e.g., a non-woven) and enter inside the spacer layer 322b (e.g., mesh-see arrow E). Within the mesh spacer layer 322b, the drag force applied is minimized (eventually to zero) and the volume of liquid can flow all along the spacer layer. As shown in FIG. 7, the slanting may be at an angle α of, for example, 30 to 45 degrees, relative to the horizontal H, and may be achieved by slanting the entire bioreactor 312 including the fixed bed 322, or just the fixed bed 322 if it can be titled within the bioreactor 312.

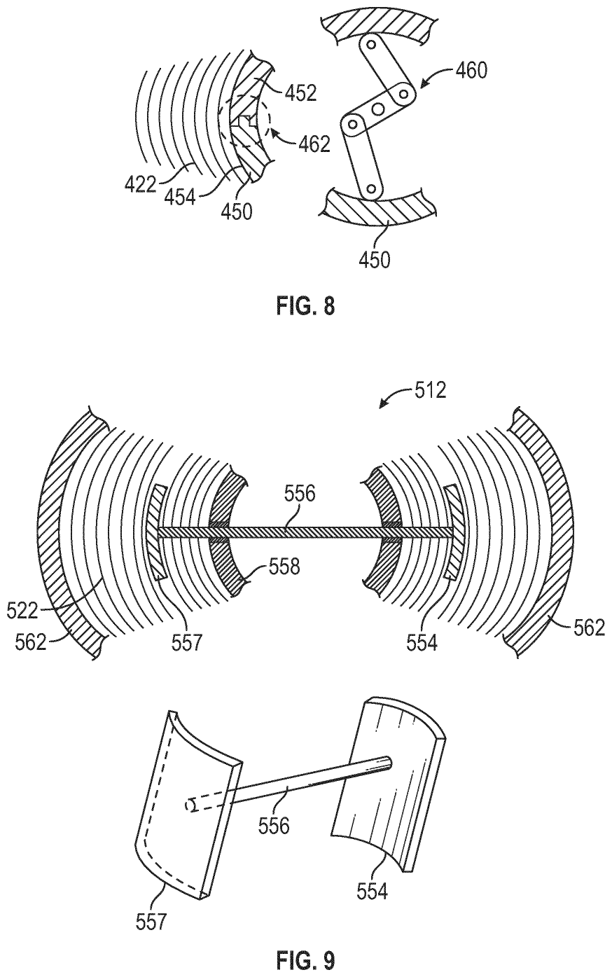

A further aspect of the disclosure for improving cell harvesting involves compressing or compacting the fixed bed. With reference to FIG. 8, this may be achieved by associating the fixed bed 422 of a bioreactor (not shown) with an internal compactor for compacting the fixed bed. In one example, the compactor may comprise a cylindrical wall 450 having interdigitated members 452, 454 capable of relative movement in a radial direction. In the illustrated version, the wall 450 is internal to the fixed bed 422, but could be external to it.

The compactor further includes an actuator for causing movement relative to the fixed bed in order to provide a compacting force to it. This actuator may comprise a linkage 460 connected to a motive device 462 (which may comprise a motor or a hand crank) for engaging the members 452, 454 and causing radial movement. As the position of the bed 422 is fixed, this movement compacts or compresses the bed 422, and thus forces the release of any retained liquid, including detached cells (either as the result of vibration, the introduction of a detachment solution, or both). The squeezing action provided may be repeated as necessary to maximize the release of liquid.

FIG. 9 illustrates another possible version of an arrangement for compressing a fixed bed 522. The arrangement may comprise opposed members 552, 554 connected to a telescoping member 556, which may include a linear actuator. The member 556 may pass through an internal wall 558 of the bioreactor 512. Actuation thus causes the members 552, 554 to push outwardly against the bed 522, squeezing it as a result of the outer wall 562 of the bioreactor 512.

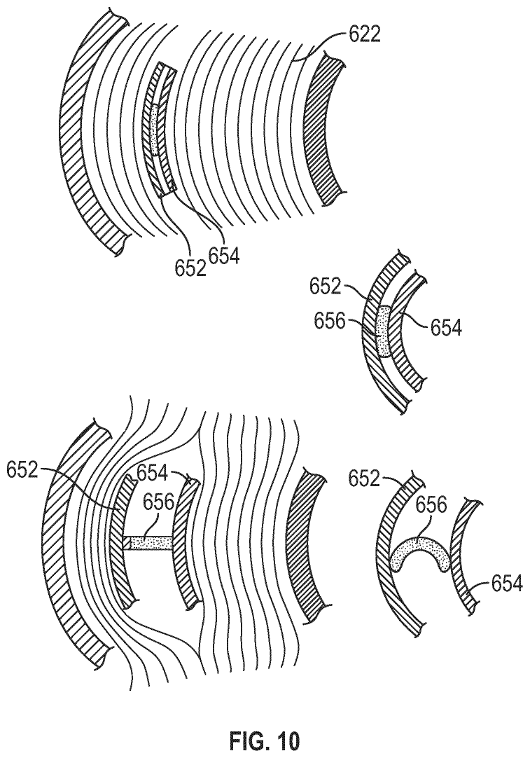

Yet another version is shown in FIG. 10. In this arrangement, opposed members 652, 654 may be mounted within the fixed bed 622 connected to an internal actuator, such as a rotatable member 656 (which may be linear or curved). Actuation of the member 656 to rotate thus forces the members 652, 654 to move apart and, as a consequence, compresses the fixed bed 622 to release the liquid therein.

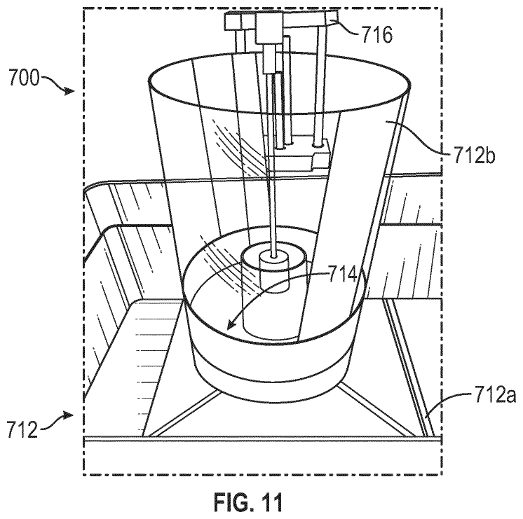

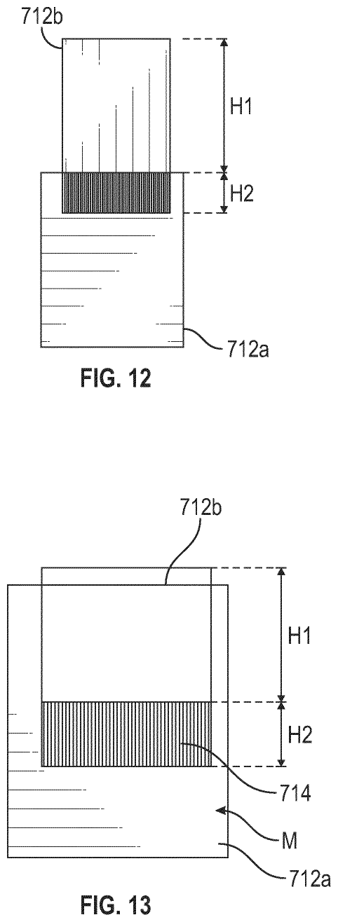

According to a further aspect of the disclosure, a bioreactor 700 may comprise a vessel 712 and including a fixed bed 714 for culturing or growing cells in connection with a fluid medium. In order to change the liquid level, the fixed bed 714 may be moved relative to the vessel 712. For example, as shown in FIGS. 11 and 12, the vessel 712 may include a main portion 712a for receiving a volume of fluid and the fixed bed 714 in one position, and an auxiliary portion 712b for receiving a portion of the volume of fluid (liquid culture medium M) and the fixed bed in a second position (the terms “main” and “auxiliary” having no bearing on shape or size of the respective portions of the vessel, although in the illustrated embodiment the auxiliary portion is shown as being smaller and cylindrical, while the main portion is larger and cubic).

The auxiliary portion 712b of the vessel 712 is adapted for receiving the fixed bed 714, and may also be adapted for moving relative to the main portion 712a of the vessel 712. Thus, as shown in FIG. 13, the auxiliary portion 712b including the fixed bed 714 may be lowered into the main portion 712a of the vessel 712, thus causing fluid to pass through the fixed bed 714 (of height H2) and into a volume (formed by height H1) of the auxiliary portion 712b above the fixed bed 714 (hence varying the overall volume of the vessel 712). Reversing the movement then causes the fluid to flow back through the fixed bed 714 and into the main portion 712a of the vessel 712. The raising and lowering may be achieved using an actuator 716, such as a linear actuator.

Using this arrangement, the fixed bed 714 remains submerged at all times, and the flow action created causes the fluid (culture medium) to pass to and fro through the fixed bed 714 in order to promote cell viability and growth. The speed of the relative (e.g., vertical) movement may be controlled in order to create a desired flow rate through the fixed bed 714, which will depend in part upon the porosity or density of the arrangement. Variability of the flow rate may also be controlled depending on the nature of the bioprocess (e.g., high flow rate to guarantee a homogeneous cell circulation or during the cell harvest or low flow rate to protect shear sensitive cells). In any case, it should be appreciated that the desired flow may be created without the use of circulation, such as by an internal agitator, to move fluid throughout the vessel, instead relying on the movement of the fixed bed 714 relative to the vessel 712.

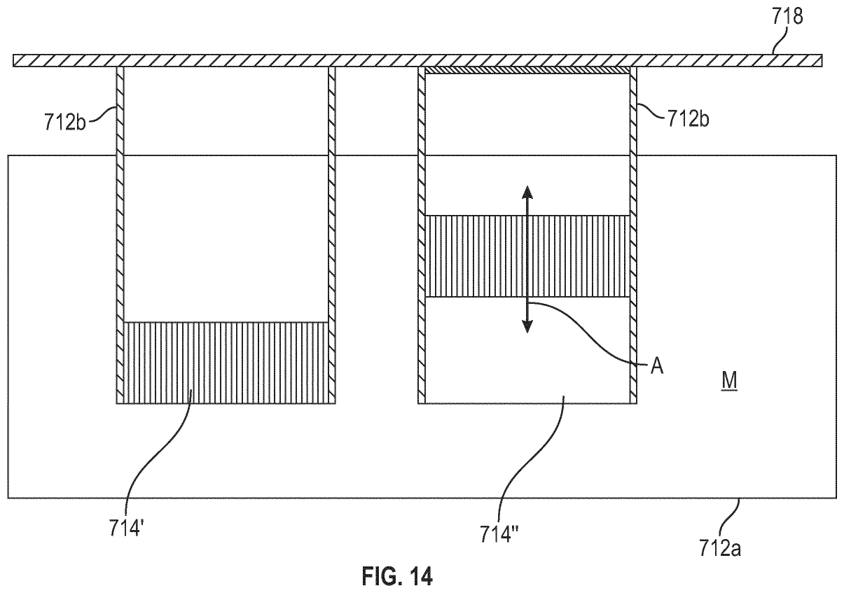

Turning to FIG. 14, an arrangement is shown schematically in which the position of the auxiliary portion 712b of the vessel 712 remains fixed relative to the main portion 712a. In this version, the fixed bed 714 is caused to move between positions within the auxiliary portion 712b (note first or lower position 714′ on the left side of FIG. 14, and second or raised position 714″ on the right side, which may be representative of the same or different auxiliary portion(s) 712b). A lid or cover 718 may also be provided, along with an actuator (not shown) for raising and lowering the fixed bed 714, which actuator may be internal to or external to the vessel 712.

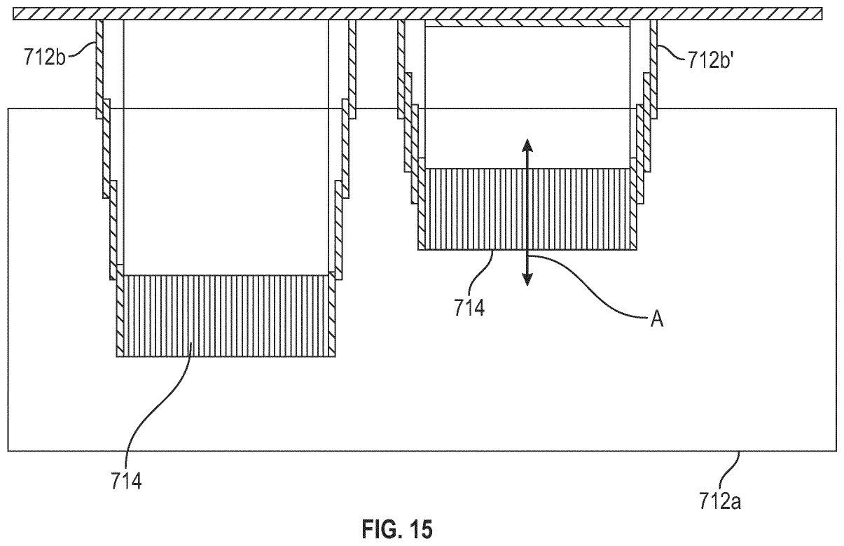

In any case, from the first position the fixed bed 714 may be raised within the auxiliary portion 712b, which will cause fluid in its path to pass through it. However, because the flow rate may be relatively slow, a portion of the fluid may be caused to flow into a previously empty space in the auxiliary portion 712b, as shown in the right hand side of FIG. 15. As a result of the connection between the portions 712a, 712b of the vessel 712, the fluid eventually reaches equilibrium, passing through the fixed bed 712 in the process. The movement of the fixed bed 714 may then be reversed, as indicated by arrow A, to move within the auxiliary portion 712b, again causing the fluid in advance of the fixed bed to pass through it during this movement. This movement may be repeated and the speed controlled to provide the desired amount of fluid (liquid culture medium M) flow to promote cell growth and viability.

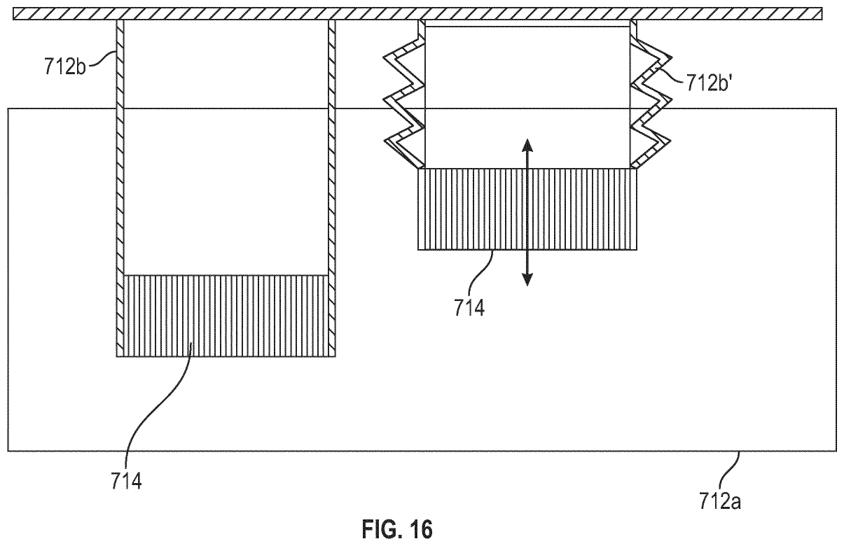

The auxiliary portion 712b described above, whether movable or not, may be formed of a generally rigid material, which may be cylindrical in nature and hollow for receiving the fixed bed. With reference to FIGS. 15 and 16, it is also possible to form the auxiliary portion 712b of the vessel of a collapsible or flexible material. Thus, as shown in FIG. 16, the auxiliary portion 712b may be telescopic, and comprise a plurality of telescoping parts (such as sliding tubes that nest together) in order to collapse and move the fixed bed 714 within the fluid of the main portion 712a of the vessel (note position 712b vs. 712b′), and also accommodate fluid withdrawn from the main portion by the movement. FIG. 16 shows an arrangement in which the auxiliary portion 712b is made flexible, like a bellows or accordion, and thus may collapse as the fixed bed 714 is moved together with the auxiliary portion 712b.

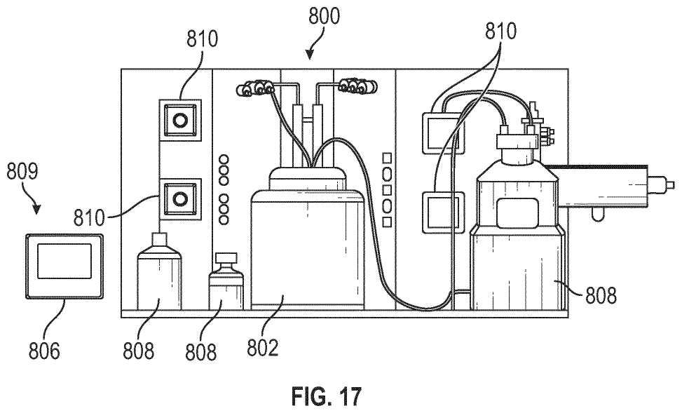

FIG. 17 illustrates a docking station 800 for a bioreactor 802. The docking station 800 may include a controller 804 having a display 806 for displaying various parameters associated with a bioprocessing operation being conducted, and also allowing for inputs to control various aspects of the same. For instance, the docking station 800 may include various auxiliary containers 808 associated with pumps 810 connected by conduits. The controller 804 may serve to control these pumps in order to control the flow of fluid to or from the bioreactor 802, as well as to control mixing of the fluid in the bioreactor such as by controlling an agitator therein (not shown), the drive for which may form part of the docking station 800.

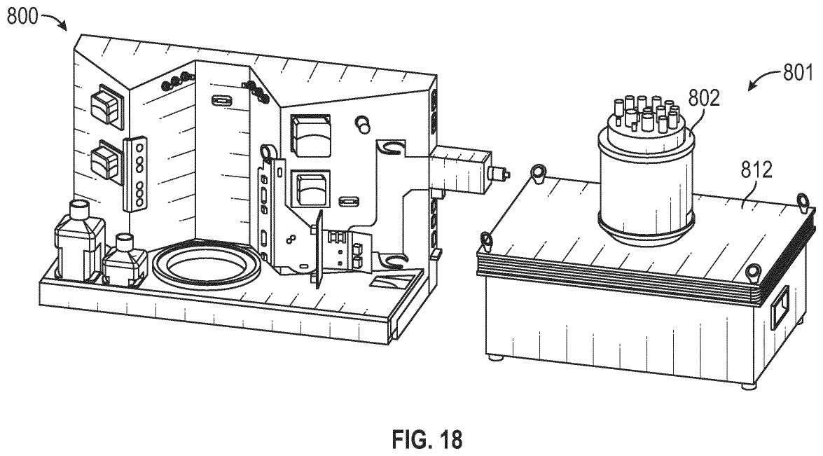

As shown in FIG. 18, the docking station 800 may be associated with a harvest module 801, including an external agitator, which is shown in the form of a vibration table 812. As indicated, the bioreactor 802 may be moved from the docking station 800 to the vibration table 812 in order to aid in harvesting cells per the teachings of this disclosure. The vibration table 812 may be independently controlled, or controller by the controller 804, and may also be integrally formed with the docking station 800.

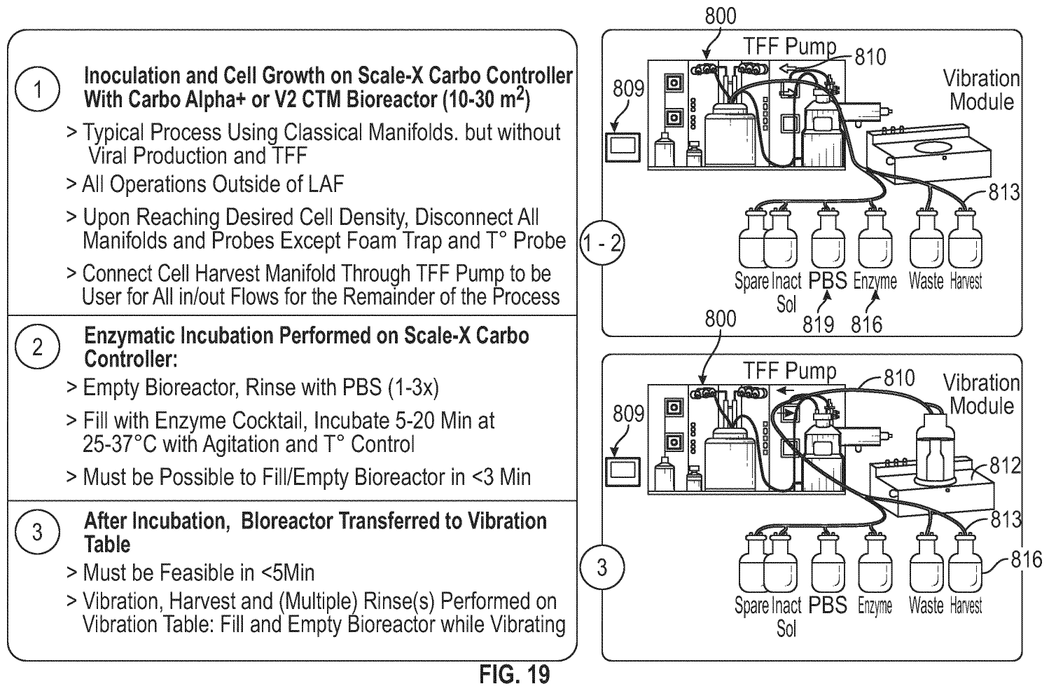

Turning to FIG. 19, the controller 804 associated with the docking station 800 may be used to control the cell harvesting operation. For example, a cell harvest manifold 813 may be connected to the bioreactor 802 once a desired cell density is reached. The bioreactor 802 may be emptied, and rinsed with buffer from a container 814, and the enzyme cocktail then introduced such as from a corresponding container 816. Pumping of the fluids to/from the bioreactor 802 may be achieved using the pumps 810 associated with the docking station 800, which may again be controlled by the controller 804.

The bioreactor 802 may then be transferred to the agitator, or vibration table 812. The vibration may be completed along with harvest to a suitable container 818. Multiple rinses may be completed, such as by using the controller 804 to control the pumping of fluid in and out of the bioreactor 802 using a pump 810 associated with the docking station 800, and in connection with the manifold 813. In this manner, the entire cell growth and harvesting process (including as disclosed herein) may be controlled by the controller 804.

### EXAMPLE

Experiments were conducted to assess the viability of the cell harvesting techniques according to this disclosure. Adherent HEK293 cells from a cryopreserved cell bank (18H003, ECACC) were used for all experiments. Plastic flatware and bioreactor cultures were performed in DMEM (4.5 g/L glucose) supplemented with 5% fetal bovine serum. Inoculation was performed in all conditions at 20,000-25,000 cells/cm². Prior to cell culture in bioreactors, cells were precultured in T-flasks and multilayer plastic vessels to reach required inoculum. Passaging was performed every 3 to 4 days (in mid-exponential phase).

Cells were inoculated in a scale-X hydro (2.4 m²), carbo 10 m² and 30 m² bioreactors and kept for 4 h in batch mode prior starting the culture growth using a recirculation loop (0.17 mL/cm²). Bioreactor culture conditions are detailed in Table 1:

TABLE 1
Bioreactor culture conditions

| Bioreactors | scale-X hydro 2.4 m² | |
|---|---|---|
| | scale-X carbo 10 and 30 m² | |
| Cell line | Adherent HEK 293 | |
| pH | 7.20 | |
| DO | >50% | |
| Temperature | 37° | C. |
| Agitation | 0.5 | cm/s |
| Feeding strategy | DMEM (4.5 g glucose/L) with | |
| | 5% FBS; Recirculation loop | |
| | with 0.17 ml/cm² | |
| Growth phase | 4-6 | days |
| Cell seeding | 20-25,000 | cells/cm² |

Daily samples of media and fixed-bed (via sampling carriers) were taken to evaluate cell growth through glucose and lactate profiles and direct cell counts on sampling carriers respectively.

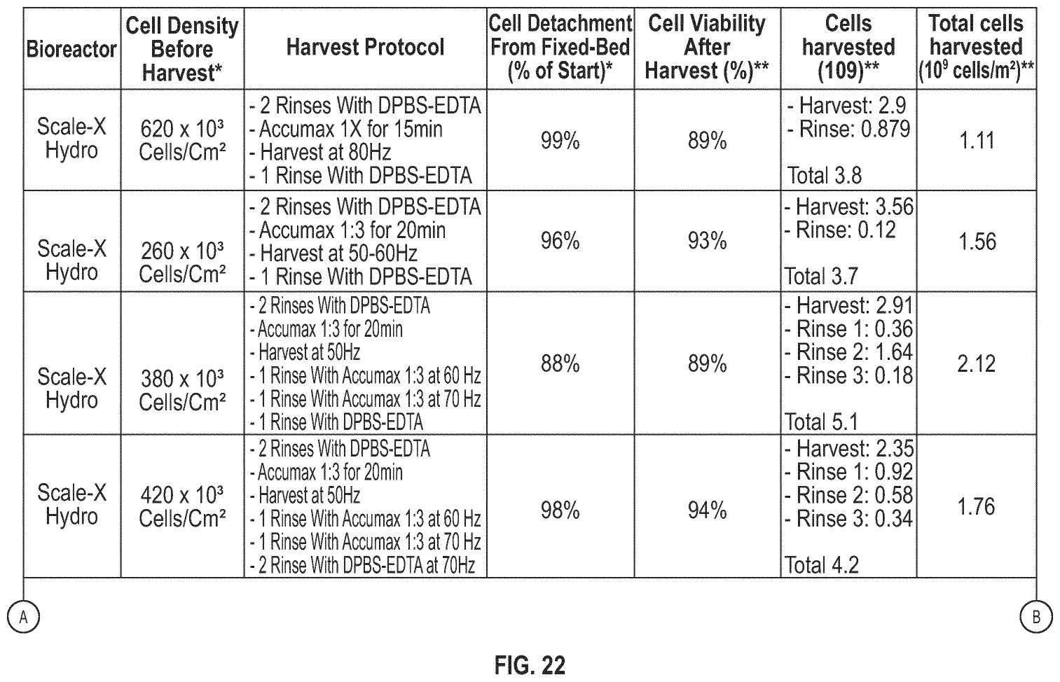

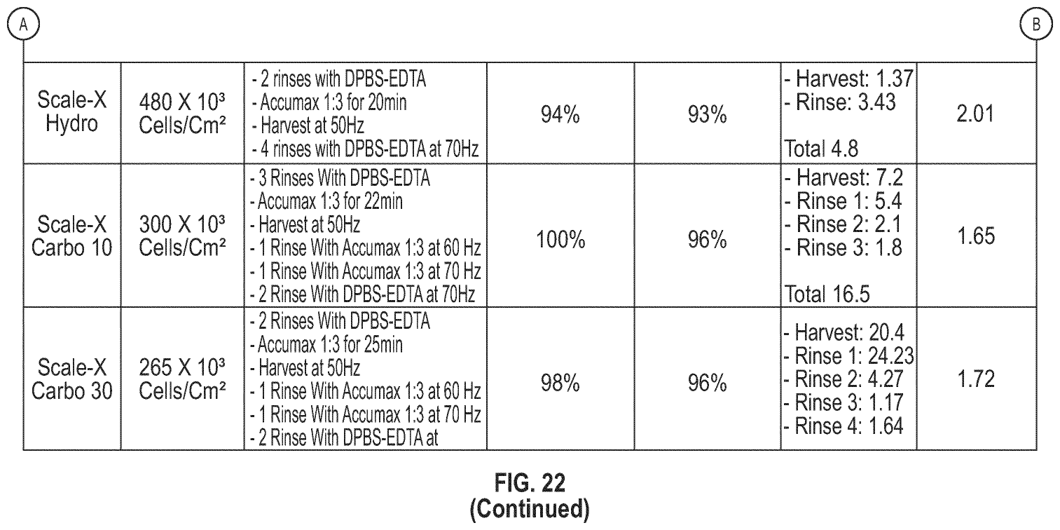

Harvest from bioreactors was performed after 4 to 6 days of expansion. Prior to being harvested, bioreactors were emptied and rinsed with a DPBS solution containing 5 mM EDTA (DPBS-EDTA), preheated at 37° C. Subsequently, detaching solution preheated a 37° C. was added to bioreactors, followed by a 20-25 min incubation with agitation (0.5 cm/s) and temperature control (37° C.). Bioreactors were then moved to the harvesting module and subjected to vibration with simultaneous vessel draining. Various combinations of vibration frequency and duration were explored and are detailed in FIG. 22.

In certain instances, multiple rinses with enzymatic solution followed the first harvest. In all cases, following harvest, bioreactors were rinsed with DPBS-EDTA. In certain cases, vibration was applied during some or all rinsing steps. Samples of fixed-bed carriers and supernatant were taken at various steps of the harvesting process to determine harvest efficiency and cell viability. For the present study, two detaching enzymatic solutions were tested: TrypLE™ Select, either 1× or 5× (Gibco), and Accumax (Innovative Cell Technologies), either 1× or diluted 1:3 in DPBS-EDTA.

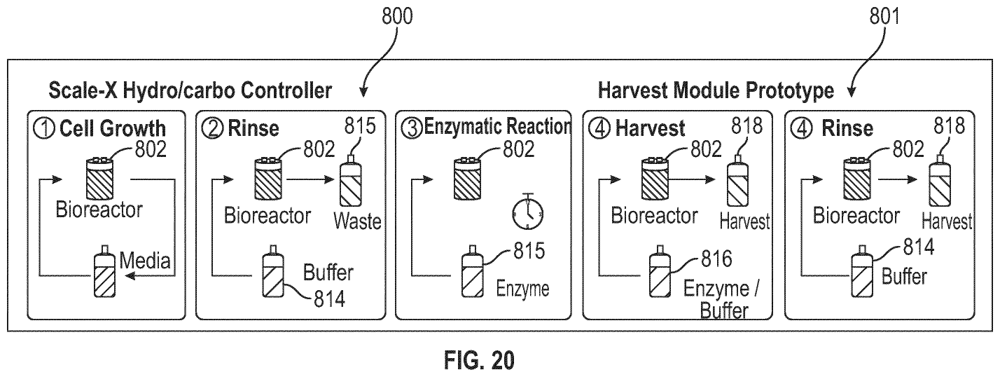

An overview of the cell harvest protocol is presented in FIG. 20. Briefly, cells were expanded in bioreactors 802 for 4-6 days (1) using media from an associated container 803 in a recirculation arrangement. On the day of harvest, bioreactors 802 were rinsed (2) using buffer from a container 814 and an associated waste vessel 815 then filled with the enzymatic solution from a vessel 816 for a 20-25 min incubation (3). Subsequently, bioreactors 802 were drained to a harvest vessel 818 with simultaneous vibration (4). Finally, rinsing using buffer 814 was performed to facilitate cell recovery (5). As can be understood, steps 1-3 were performed with bioreactors 802 installed on a controller 800. Steps 4-5 were performed with bioreactors 802 installed on the harvest module 801.

The cell density and viability in harvested cells was measured by Trypan Blue dye exclusion method using a hemocytometer. The biomass estimation on the sampling carriers were performed by cell lysis (scale-X cell counting kit, Univercells Technologies) followed by a crystal violet staining and cell nuclei counting with a hemocytometer. Cell nuclei counts on fixed-bed fibers were used to estimate the total cell number inside the bioreactor. After cell harvest, bioreactors were dismantled and portions of the fixed-bed were taken from representative locations to assess the remaining cell density on the fixed-bed.

Cells harvested from scale-X hydro (n=5), carbo 10 (n=1) and 30 (n=1) bioreactors were seeded in T-flasks to assess replating efficiency, cell morphology and cell recovery on the first passage. Population doubling time (PDT) and cell morphology were assessed in comparison to control cells harvested from plastic flatware. In one case, cells harvested from a scale-X hydro bioreactor were used to seed a second hydro to provide proof-of-concept for a “fixed-bed to fixed-bed” seed train. Inoculation conditions using hydro-harvested cells were identical as described previously.

In a first phase, tests were performed using TrypLE 1× and 5× as detaching solution. However, the conditions tested were prone to release cells with a large proportion of aggregates, thereby affecting total cell recovery (data not shown). In contrast, the use of Accumax was found to produce single cell suspensions in the conditions tested. All experiments described in the present document were thus performed with Accumax, either 1× or diluted to a 1:3 solution.

Five cell harvest experiments were performed with the standard version of the scale-X hydro. In all experiments, Accumax incubation was followed by a harvest step followed by several rinsing steps as explained in the Materials and Methods section. Assessment of different harvest methodologies indicated that 1 harvest cycle followed by 2 rinsing cycles using Accumax, all accompanied with vibrations between 50-70 Hz, were most efficient in recovering cells (FIG. 22). Additional rinsing steps with DPBS-EDTA enabled moderate additional cell release. Applying vibration during rinsing further increased the amount of cells recovered.

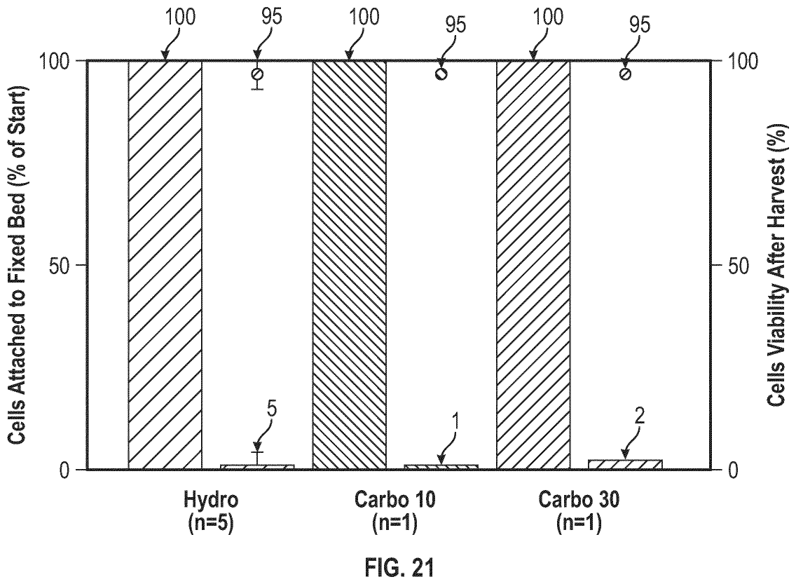

In all experiments, analysis of sampling carriers before and after harvest indicated >90% of cells were detached from the fixed-bed material (FIGS. 21-22). In total, between 2.7 and 5.1×10⁹ cells could be harvested from scale-X hydro bioreactors in the form of a single cell suspension. Performing one harvest cycle after Accumax incubation, followed by two rinsing cycles using Accumax, all accompanied with vibrations between 50-70 Hz, conducted to the highest single cell harvest yield. Cells could be harvested from bioreactors both at mid-exponential phase (200-300×10³ cells/cm²) and closer to confluence (400-500×10³ cells/cm²), indicating that optimizing cell number upon harvest may be critical to achieve the highest harvest yields. Of note, cell harvest in plastic flatware is typically performed at cell densities of 200×10³ cells/cm², highlighting an advantage of fixed-bed bioreactors enabling cell harvest at higher densities.

Following proof-of-concept and optimization at hydro scale, additional cell harvest experiments were performed to investigate scalability to the carbo 10 (n=1) and 30 bioreactors (n=1). In both harvests, >98% of cells were detached from the fixed-bed material. As much as 51×10⁹ cells were collected from the scale-X carbo 30 in the form of a single cell suspension, therefore providing enough cells for inoculation of a scale-X nitro 600 at 8,500 cells/cm². As shown in hydro experiments, cell densities of up to 450×10³ cells/cm² could be efficiently harvested, which in carbo 30 would translate to the ability of seeding a nitro 600 at 10,000 cells/cm 2. Of note, in the two experiments with carbo, a manual upside-down step was added to facilitate bioreactor draining and comparison with scale-X hydro data. It is estimated is that a full harvest without this manipulation would require two additional rinsing steps than listed in the present study (five in total).

In all experiments performed for the present study (hydro, carbo 10 and 30), harvested cells presented excellent viability (between 84 and 96%) (FIG. 21-22). In runs with diluted Accumax, no aggregates were visible in the collected suspension.

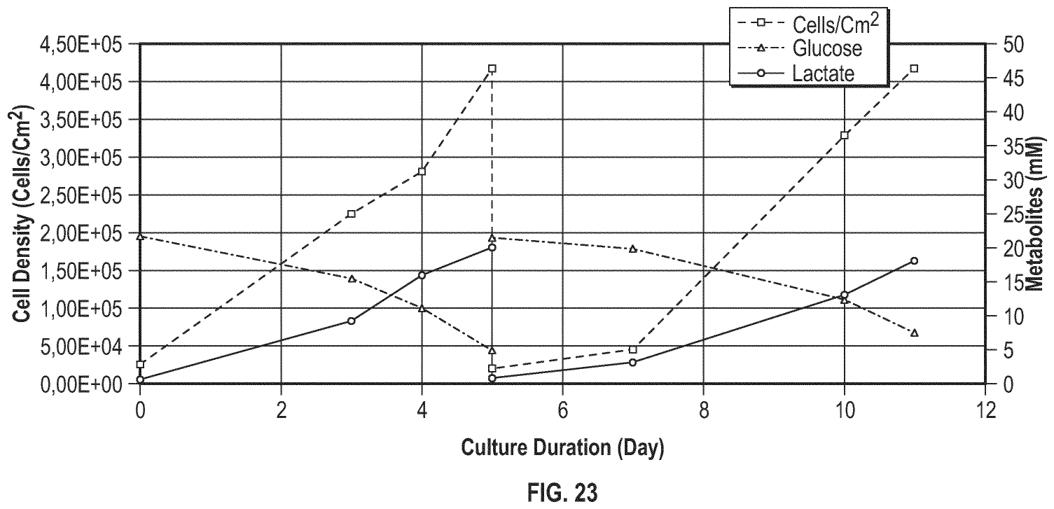

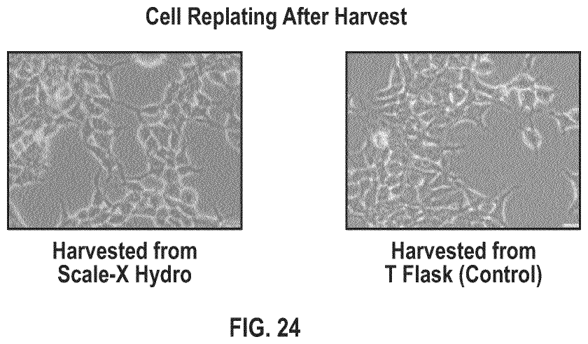

Replating experiments were performed with cells collected at all scale, indicating excellent replating in T-flasks with PDTs of 29-37 h (compared to 29-39 h with control cells related from T-flasks; FIG. 23. In one case, cells harvested from a scale-X hydro were used to inoculate a second hydro, where appropriate growth was observed (FIG. 24). Cells could then be harvested from the second hydro, providing proof-of-concept for a fixed-bed to fixed-bed seed train.

The above results demonstrate that the harvest from a scale-X carbo 30 m² should be sufficient to inoculate a scale-X nitro 600 m² at 10,000 HEK293 cells/cm². Importantly, a scale-X carbo 30 is equivalent to twelve 40-layers plastic flatware vessels, which are typically operated with the use of specialized equipment such as large incubators, automated manipulators and shakers. Simple estimations comparing a seed train process using plastic flatware to a scale-X carbo show a drastic reduction (95%) in equipment footprint with the latter.

In the conditions tested for the present study, multiple rinses with Accumax were required to obtain a proper cell recovery. Despite this need for multiple rinses, it is estimated that total enzyme use to remain lower compared to traditional plastic flatware processes (9.0 L for scale-X carbo 30 m² vs 9.6 L for 12×CF40). In addition, it is showed that it is feasible to reduce the amount of enzyme per surface with some optimization.

Considering the volume harvested, scale-X 30 m² conducts to high inoculum cell density and lower volume of seed: 15 L (3 harvest of 3 L and 2 rinse of 3 L) than classical flatware: 28.8 L for 12 CF-40 (considering the recommended following volumes: 20 ml enzyme+25 ml of media and 15 ml of rinse per layer)

The scale-X bioreactor could be harvested in closed-system by a single operator within 2 h, while harvesting cells from multiples CF-40 required more operators and aseptic connections under LAF.

While these initial estimates clearly point to significant cost savings for seed train generation using the scale-X carbo, more complete cost modeling is likely to display even larger differences due to labor, total operating footprint, aseptic risk, media use etc.

Summarizing, this disclosure may be considered to relate to any or all of the items in any combination or arrangement:

1. A method of harvesting cells, comprising:
providing a bioreactor including a fixed bed structure capable of cell entrapment or adherence and cell growth;
adding cells to the bioreactor via media;
allowing the cells to become entrapped and/or adhere to the fixed bed structure and grow within the bioreactor;
introducing a cell detaching solution comprising an enzymatic cocktail into the bioreactor;
agitating a portion of the bioreactor; and
moving a liquid level of the cell detaching solution relative to the fixed bed structure; wherein a substantial portion of the cells are detached from the fixed bed structure without forming clumps or aggregates in the substantial portion of the cells.

2. The method of item 1, wherein the agitating and moving steps are done simultaneously.

3. The method of item 1 or item 2, wherein the moving step comprises at least partially draining the bioreactor of the cell detaching solution.

4. The method of any of items 1-3, wherein the moving step comprises moving the liquid level from above or proximate a top of the fixed bed structure to below or proximate a bottom of the fixed bed.

5. The method of any of items 1-4, wherein the moving step comprises adding fluid to the bioreactor.

6. The method of any of items 1-5, wherein the adding step comprises adding additional cell detaching solution to the bioreactor.

7. The method of any of items 1-6, wherein the liquid level is located above the fixed bed structure prior to the moving step.

8. The method of any of items 1-7, wherein the moving step comprises raising and lowering the liquid level a plurality of times.

9. The method of any of items 1-8, wherein the agitating step comprises directly or indirectly vibrating the fixed bed, such as at a frequency of between 20 and 300 Hertz and with an amplitude between 0.5 and 5 millimeters.

10. The method of any of items 1-9, wherein the step of introducing comprises introducing enzymes that cleave integrins and different enzymes that cleave an extracellular matrix as the enzymatic cocktail.

11. A system for harvesting cells, comprising:
a bioreactor including a structure for cell entrapment/adherence and growth;
a cell harvest mechanism adapted to agitate the bioreactor and to move a liquid level relative to the structure; and
a vessel including a cell detaching solution in fluid communication with the bioreactor, the cell detaching solution comprising an enzymatic cocktail for the detachment of cells from the structure for cell entrapment/adherence and growth without producing clumps or aggregates.

12. The system of item 11, wherein the structure for cell entrapment/adherence and growth comprises a fixed bed, such as a 3-D printed fixed bed.

13. The system of item 11 or item 12, wherein the structure for cell entrapment/adherence and growth comprises a fixed bed having a plurality of cell immobilization layers, such as arranged in a stack or a spiral configuration, and either in direct contact or with a spacing between adjacent layers.

14. The system of any of items 11-13, wherein the cell harvest mechanism comprises a device for vibrating or shaking the bioreactor.

15. The system of any of items 11-14, wherein the cell harvest mechanism comprises a pump for moving the liquid level.

16. The system of any of items 11-15, wherein the cell harvest mechanism comprises a device for applying vibratory energy to the bioreactor and, in particular, to the structure for cell entrapment/adherence and growth.

17. The system of any of items 11-16, wherein the cell harvest mechanism forms part of a docking station for the bioreactor.

18. The system of any of items 11-17, wherein the bioreactor comprises a harvest vessel for harvesting cells for introduction to another bioreactor.

19. The system of any of items 11-18, wherein the bioreactor is tilted relative to a horizontal plane to facilitate draining of liquid from the structure for cell entrapment/adherence and growth.

20. The system of any of items 11-19, further including a compactor for compacting the structure for cell entrapment/adherence and growth, either internal or external thereto.

21. The system of any of items 11-20, wherein the enzymatic cocktail includes enzymes that cleave integrins and different enzymes that cleave an extracellular matrix.

22. The system of any of items 11-21, wherein the cell harvest device comprises an actuator for moving the structure for cell entrapment/adherence and growth relative to the bioreactor to move a location of the liquid level.

23. The system of any of items 11-22, further including a controller for controlling the cell harvest mechanism to agitate the bioreactor and move a liquid level relative to the structure for cell entrapment/adherence and growth.

24. The system of any of items 11-23, wherein the controller is adapted for controlling delivery of the enzymatic cocktail to the bioreactor.

25. A system for harvesting cells, comprising:
a bioreactor including a structure for cell entrapment/adherence and growth;
an agitator adapted to agitate the bioreactor;
an actuator for moving a liquid level relative to the structure for cell entrapment/adherence and growth; and
a vessel including a cell detaching solution in fluid communication with the bioreactor, the cell detaching solution comprising an enzymatic cocktail for the detachment of cells from the structure for cell entrapment/adherence and growth without producing clumps or aggregates.

26. The system of item 25, wherein the agitator comprises a vibrator.

27 The system of item 25 or item 26, wherein the actuator comprises a linear actuator.

28. The system of any of items 25-27, wherein the actuator comprises a pump.

29. The system of any of items 25-28, further including a controller for controlling the actuator.

30. A system for harvesting cells, comprising:
a bioreactor including a structure for cell entrapment/adherence and growth;
a cell harvest mechanism adapted to adapted to agitate the bioreactor while filling and flushing the bioreactor with liquid;
a vessel including a cell detaching solution in fluid communication with the bioreactor.

31. The system of item 30, wherein the cell detaching solution comprises an enzymatic cocktail for the detachment of cells without producing clumps or aggregates.

32. The system of item 30 or item 31, wherein the cell harvest mechanism comprises a device for vibrating or shaking the bioreactor.

33. The system of any of items 30-32, wherein the cell harvest mechanism comprises a device to partially or completely fill, empty and flush the bioreactor.

34. The system of item 33, wherein the fill, empty and flush device comprises one or more pumps.

35. The system of any of items 30-34, wherein the cell harvest mechanism comprises a device for applying vibratory energy to the bioreactor and, in particular, to the structure for cell entrapment/adherence and growth.

36. The system of any of items 30-35, wherein the cell harvest mechanism forms part of a docking station for the bioreactor.

37. A method of detaching cells from a fixed bed bioreactor, comprising:
adding to the fixed bed bioreactor an enzymatic cocktail for the detachment of cells without producing clumps or aggregates; and
adjusting a position of a liquid level in the fixed bed bioreactor while vibrating the bioreactor.

38. The method of item 37, wherein the adjusting step comprises filling and flushing the bioreactor with liquid.

39. The method of item 36 or item 37, wherein the adjusting step comprises repeatedly filling and flushing the bioreactor with liquid.

40. The method of any of items 37-39, further including the step of delivering detached cells from the bioreactor to another bioreactor.

41. The method of any of items 37-40, further including the step of tilting the bioreactor.

42. The method of any of items 37-41, further including the step of compacting a fixed bed in the bioreactor.

43. The method of any of items 37-42, wherein the adjusting step comprises moving the fixed bed relative to the bioreactor.

44. A method of detaching cells in a bioreactor, comprising:
vibrating the bioreactor; and
tilting and draining the bioreactor.

45. The method of item 44, wherein the vibrating, tilting, and draining steps are performed concurrently.

46. A system for harvesting cells, comprising:
a bioreactor including a fixed bed for adherent cell growth; and
a compactor for compacting the fixed bed.

47. The system of item 46, further including a vibrator for vibrating the bioreactor or the fixed bed.

48. The system of item 46 or item 46, wherein the compactor is located within the fixed bed or external to the fixed bed.

49. A system for harvesting cells, comprising:
a preculture vessel including a structure for adherent cell growth;
a vibrator adapted to vibrate the bioreactor in order to detach cells from the structure; and
a bioreactor downstream of the preculture vessel for receiving the detached cells.

50. The system of item 49, further including a pump for pumping liquid to or from the preculture vessel so to move a liquid level relative to the structure.

51. The system of item 50, further including a controller for controlling the pump.

52. The system of item 51, wherein the controller is adapted for controlling the vibrator.

For purposes of this disclosure, the following terms have the following meanings:

“A”, “an”, and “the” as used herein refers to both singular and plural referents unless the context clearly dictates otherwise. By way of example, “a compartment” refers to one or more than one compartment.

“About,” “substantially,” “generally” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, “comprises” and “comprised of” as used herein are synonymous with “include”, “including”, “includes” or “contain”, “containing”, “contains” and are inclusive or open-ended terms that specifies the presence of what follows, e.g., “component includes” does not exclude or preclude the presence of additional, non-recited components, features, element, members, steps, known in the art or disclosed therein.

While preferred embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the protection under the applicable law and that methods and structures within the scope of these claims and their equivalents be covered thereby.

1. A method of harvesting cells, comprising:
providing a bioreactor including a fixed bed structure capable of cell entrapment or adherence and cell growth;
adding cells to the bioreactor via media;
allowing the cells to become entrapped and/or adhere to the fixed bed structure and grow within the bioreactor;
introducing a cell detaching solution comprising an enzymatic cocktail into the bioreactor;
agitating a portion of the bioreactor; and
moving a liquid level of the cell detaching solution relative to the fixed bed structure;
wherein a substantial portion of the cells are detached from the fixed bed structure without forming clumps or aggregates in the substantial portion of the cells.

2. The method of claim 1, wherein the agitating and moving steps are done simultaneously.

3. The method of claim 1, wherein the moving step comprises at least partially draining the bioreactor of the cell detaching solution.

4. The method of claim 1, wherein the moving step comprises moving the liquid level from above or proximate a top of the fixed bed structure to below or proximate a bottom of the fixed bed.

5. The method of claim 1, wherein the moving step comprises adding fluid to the bioreactor.

6. The method of claim 1, wherein the adding step comprises adding additional cell detaching solution to the bioreactor.

7. The method of claim 1, wherein the liquid level is located above the fixed bed structure prior to the moving step.

8. The method of claim 1, wherein the moving step comprises raising and lowering the liquid level a plurality of times.

9. The method of claim 1, wherein the agitating step comprises directly or indirectly vibrating the fixed bed, such as at a frequency of between about 20 and about 300 Hertz and with an amplitude between about 0.5 and about 5 millimeters.

10. The method of claim 1, wherein the step of introducing comprises introducing enzymes that cleave integrins and different enzymes that cleave an extracellular matrix as the enzymatic cocktail.

11. A system for harvesting cells, comprising:
a bioreactor including a structure for cell entrapment/adherence and growth;
a cell harvest mechanism adapted to agitate the bioreactor and to move a liquid level relative to the structure; and
a vessel including a cell detaching solution in fluid communication with the bioreactor, the cell detaching solution comprising an enzymatic cocktail for the detachment of cells from the structure for cell entrapment/adherence and growth without producing clumps or aggregates.

12. The system of claim 11, wherein the structure for cell entrapment/adherence and growth comprises a fixed bed, such as a 3-D printed fixed bed.

13. The system of claim 11, wherein the structure for cell entrapment/adherence and growth comprises a fixed bed having a plurality of cell immobilization layers, such as arranged in a stack or a spiral configuration, and either in direct contact or with a spacing between adjacent layers.

14. The system of claim 11, wherein the cell harvest mechanism comprises a device for vibrating or shaking the bioreactor.

15. The system of claim 11, wherein the cell harvest mechanism comprises a pump for moving the liquid level.

16. The system of claim 11, wherein the cell harvest mechanism comprises a device for applying vibratory energy to the bioreactor and, in particular, to the structure for cell entrapment/adherence and growth.

17. The system of claim 11, wherein the cell harvest mechanism forms part of a docking station for the bioreactor.

18. The system of claim 11, wherein the bioreactor comprises a harvest vessel for harvesting cells for introduction to another bioreactor.

19. The system of claim 11, wherein the bioreactor is tilted relative to a horizontal plane to facilitate draining of liquid from the structure for cell entrapment/adherence and growth.

20. The system of claim 11, further including a compactor for compacting the structure for cell entrapment/adherence and growth, either internal or external thereto.

21-52. (canceled)
